(19) United States
(12) Patent Application Publication
(10) Pub. No.: US 2004/0059194 A1
(43) Pub. Date: Mar. 25, 2004

(54) Method and apparatus for replacing knee-joint

(75) Inventors: Christopher Lee Berg, Blaine, MN (US); Todd William Sharratt, Birchwood, MN (US); Steven Mark LeVahn, Lino Lakes, MN (US)

Correspondence Address: Z. Peter Sawicki, Westman, Champlin & Kelly, Suite 1600, 900 Second Avenue South, Minneapolis, MN, 55402-3319, US

(73) Assignee: Minnesota Scientific, Inc., St. Paul, MN (US)

(21) Appl. No.: 10/623,179

(22) Filed: Jul. 18, 2003

Related U.S. Application Data
(60) Provisional application No. 60/396,850, filed on Jul. 18, 2002.

(51) Int. Cl. A61B 1/32

(52) U.S. Cl. 600/227

(57) ABSTRACT

The present invention includes a method of performing knee-joint replacement surgery. The tibia and the femur are positioned such that the knee-joint is disposed in a bent position. An incision is made over or adjacent the patella to expose the knee-joint. A retractor support is extended along at least opposite sides of the knee-joint. The skin flesh layers proximate the knee-joint are retracted utilizing a plurality of retractors which are attached to the retractor support. At least one of the retractors is attached to the retractor such that the retractor may be flexed sufficiently for movement of the knee-joint during surgery without having to reposition the retractors.

The present application is based on and claims the benefit of U.S. provisional patent application Serial No. 60/396,850, filed Jul. 18, 2002, the content of which is hereby incorporated by reference in its entirety.

### BACKGROUND OF THE INVENTION

The present invention relates to a method for performing knee-joint replacement surgery and apparatus for use in such surgery.

The knee-joint is the largest and the most complex joint in the body. The knee joint has four main parts consisting of the lower femur, the upper tibia, cartilage separating the lower femur and the upper tibia and the patella which is commonly known as the kneecap. When the knee-joint functions properly, the upper end of the tibia and the lower end of the femur glide with respect to each other and allow the knee to bend. The cartilage separates the lower end of the femur and upper end of the tibia and provides cushioning between the tibia and femur similar to a shock absorber. The surfaces which are not in contact with the cartilage are covered by a thin smooth tissue liner called the synovial membrane which releases a special fluid that lubricates the knee and reduces the friction in the knee to nearly zero in a healthy knee.

The most common cause of chronic knee pain is arthritis of which osteoarthritis, rheumatoid arthritis and post traumatic arthritis are the most common forms. Osteoarthritis typically occurs after the age of 50 and is caused by the softening and wearing away of the cartilage. As the cartilage is worn away, the tibia and femur rub against each other which causes pain and stiffness.

The second type of arthritis is rheumatoid arthritis which causes the synovial membrane to become thickened and inflamed, producing excessive amounts of synovial fluid which over-fills the joint space. The chronic inflammation can damage the cartilage and eventually cause cartilage loss, pain and stiffness.

The third type of arthritis is post traumatic arthritis which follows a serious knee injury. A knee fraction or severe tear of the knee ligaments may damage the cartilage over time. The damage to the cartilage causes pain and stiffness in the knee joint.

The arthritis in the knee can become painful to the point of extremely limiting the mobility of the person. When medications such as analgesics cannot eliminate or make the pain manageable, an increasingly popular option is to have a total knee replacement operation where the damaged knee joint is replaced with an artificial knee-joint called a prosthesis.

The current procedure for performing a total knee replacement surgery is very taxing on the surgical personnel. An incision is made from the top of the knee exposing the patella. A retractor is disposed into the incision and to one side of the patella. The surgical personnel manually retract the patella to one side and manually use additional retractors to retract the flesh to expose the femur and tibia.

With the femur and tibia exposed, the joint is separated to gain access to either the end of the femur or tibia typically by adjusting the position of the tibia. The ends of the femur and tibia are precisely cut and inserts are attached to each end of the bones. Typically, a metal piece made of stainless steel or titanium is inserted into the femur and an insert made of a durable, non-wearing plastic, typically polyethylene, is inserted into the tibia. The interface of the metal and the plastic provides a smooth moving joint that does not require lubrication. To gain access to the ends of the bones requires manipulation of the shin portion of the leg and the thigh portion of the leg which requires additional personnel.

### SUMMARY OF THE INVENTION

The present invention includes a method of performing knee-joint replacement surgery. With the patient lying on a surgical table, the tibia and the femur are positioned to place the knee-joint in a bent position. An incision is made over or adjacent the patella to expose the knee-joint. A retractor support which is mounted onto the surgical table is extended along opposite sides of the knee-joint. Skin and flesh layers proximate the knee-joint are retracted utilizing a plurality of retractors which are attached to the retractor support. At least one of the retractors that is attached to the retractor support has a portion which is flexible such that the knee-joint may be moved from an initial selected position to a second selected position during the procedure without having to reposition the retractor blades from their original engagement of the skin and the flesh layers or reattach the retractors to the retractor support or reposition the retractor support.

### BRIEF DESCRIPTION OF THE DRAWINGS

FIG. 1 is a perspective view of the apparatus used in the surgical procedure of the present invention.

FIG. 2 is a perspective view of an alternative apparatus used in the surgical procedure of the present invention.

FIG. 3 is a side view of a surgical retractor for use in the method of the present invention.

### DETAILED DESCRIPTION OF THE PREFERRED EMBODIMENTS

The present invention includes a method and apparatus for performing knee-joint replacement surgery in a manner that does not require repositioning of the surgical retractors during the knee-joint replacement surgery.

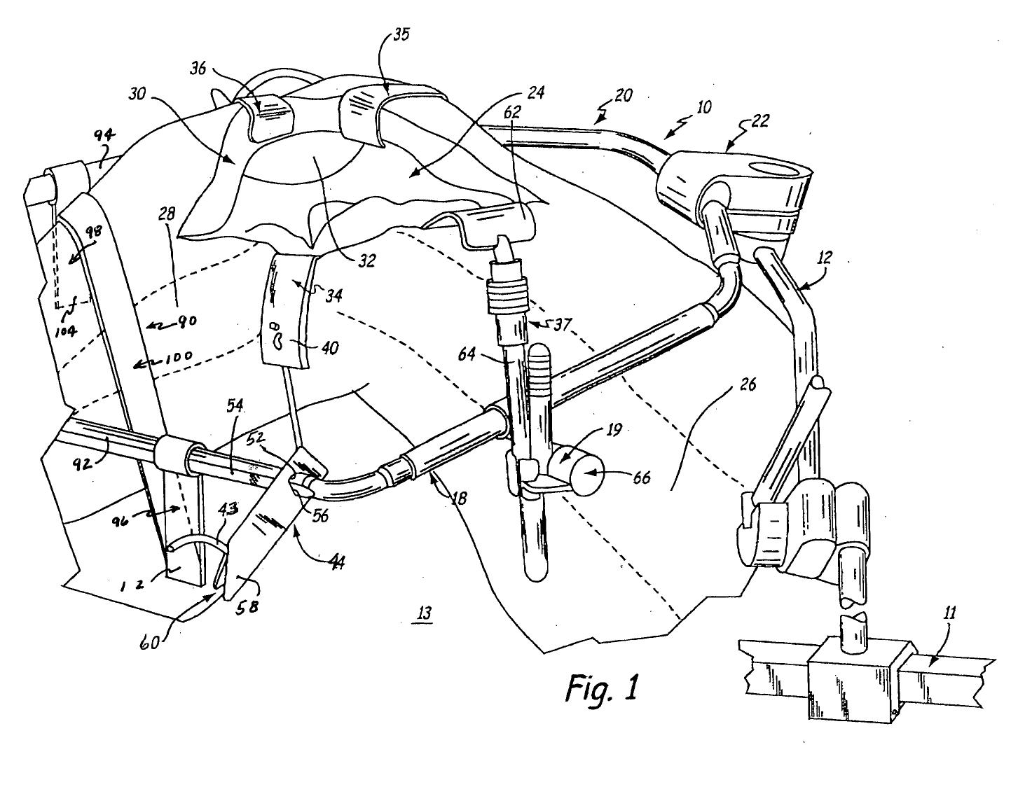

The apparatus used in the knee-joint replacement surgery of the present invention is generally indicated at 10 in FIG. 1. The apparatus 10 includes a retractor support apparatus 12 that is rigidly mounted to a rail 11 of a surgical table 13 in a manner that is well known in the art and is described in U.S. Pat. Nos. 4,617,916, 4,718,151, 4,949,707, 5,400,772, 5,741,210, 6,042,541, 6,264,396 and 6,315,718 all of which are herein incorporated by reference. From the mount to the surgical table, the retractor support apparatus 12 includes first and second support arms 18 and 20 that extend over the surgical table. The support arms 18 and 20 are independently adjustable into an infinite number of selected positions through use of a clamping mechanism 22 which is described in U.S. Pat. Nos. 5,899,627 and 6,264,396, which are herein incorporated by reference. The support arms 18 and 20 extend in a generally lateral or horizontal direction on opposite sides of a knee-joint 24. The clamp 22 secures the adjustable support arms 18 and 20 in selected angular positions with respect to the knee-joint 24.

The knee-joint 24 is preferably placed in and supported in a bent position as is typically done in knee-joint replacement surgery. The bent position is approximately a 45° angle between the femur 26 and the tibia 28. The support arms 18 and 20 are disposed on both sides and below the knee-joint 24. An incision 30 is made on top of the knee to gain access to the joint 24. The incision is made directly over the patella 32 or on occasion to the left or right of the patella depending on the surgeon's preference.

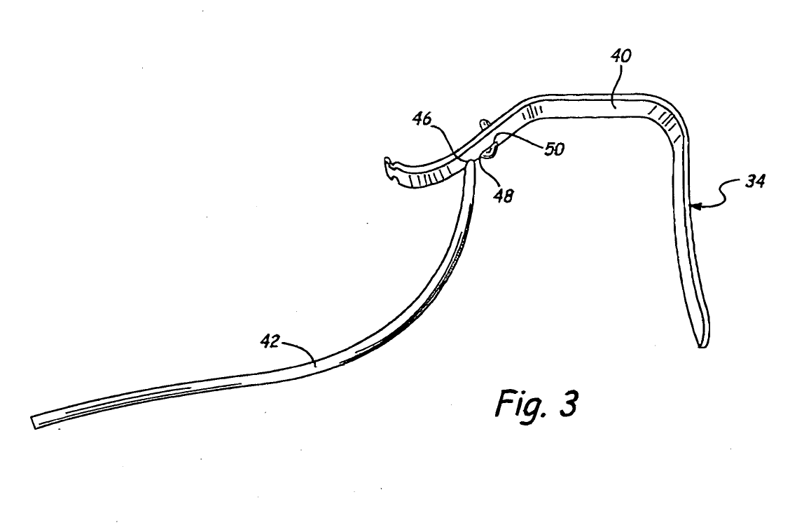

Once the incision 30 is made, a plurality of retractors 34, 35 and 36 and 37 are positioned to retract skin and flesh layers to expose the knee-joint 24. Since both surgical retractors 34 and 36 are of the same construction only retractor 34 will be described in detail. As best illustrated in FIG. 3, the surgical retractor 34 includes a retractor blade 40 attached to a flexible connector 42 such as plastic cord. The flexible connector 42 is connected to the support arm 18 by an attaching device 44.

It should be understood that although a cord is illustrated other types of flexible connectors may be used in the method of the present invention. What is important is that the retractor starting from its attachment to either support arm 18 or 20 to the skin and flesh layers not be rigid. The procedure of the present invention permits the tibia 28 or femur 26 to be moved in relation to each other without necessitating repositioning of the retractor blade, repositioning the attachment of the retractor to the retractor support or moving (adjusting) the retractor support. The flexible connector also needs to have sufficient integrity and strength to retain the retractor blade in a flesh retracted position. Although the flexible connector as shown extends from the retractor blade 40 to the attaching device 44, the flexible connector does not necessarily have to extend from the blade 40 to the device 44. For example, only a portion of the flexible connector could be flexible while the remainder could be rigid as long as sufficient flexibility exists between the retractor blade 40 and the support arm 18 or 20 to be able to reposition the tibia 28 in relation to the femur 26. For example, the flexible connector 42 may also be elastic or be made of resilient material as long as the connector is flexible. By flexible is meant that the surgeon may adjust the position of the knee-joint during surgery without having to reposition the retractor blade, reattach the retractor to the retractor support or adjust the position of the retractor support.

It is preferred that at least one of the retractors includes a flexible connector. As illustrated in FIG. 1, standard rigid retractors 37 without a flexible connector can also be used in the surgical procedure of the present invention. In other words, not all of the retractors used in the method of the present invention need to have a flexible connector. Surgical retractors which are rigid are well known in the art and are secured to the support arm 18 by a clamping mechanism 19 that is also well known. A rigid retractor 37 can be used as long as the surgeon can adjust the position of the knee-joint during surgery without having to reposition the retractor blade, reattach the retractor to the retractor support or adjust the position of the retractor support.

The flexible connector 42 is typically made of a polymeric material in the form of a solid cord. However, the connector 42 may be of any construction such as woven, braided, non-woven material or flexible metal. The flexible connector 42 is frictionally attached to the retractor blade 40 by extending through a series of holes 46, 48 and 50 in a serpentine fashion.

Knee-joint replacement surgery due to the unique positioning of the knee and its relatively light weight has posed a problem in terms of retraction of the skin and flesh. Table mounted retractors have been used for surgery on various areas of the torso. However, the torso lies flat on the surgical table and is of sufficient weight that rigid surgical retractors pulling up from an elevated position do not move the torso. However, a knee-joint must be positioned in a bent and elevated position generally supported underneath. The knee-joint also does not have the weight of a torso. Consequently retraction of the knee-joint has required significant manual assistance for proper retraction. Utilizing the procedure of the present invention by securing retractors to a table mounted support eliminates the need for additional surgery personnel to manually assist for proper retraction in holding the incision open.

As best illustrated in FIG. 1, the retractor blade 40 is positioned to engage and retract flesh along the incision 30. The flexible connector 42 is then pulled to engage the attaching device 44. As the flexible connector 42 is pulled, the retractor blade retracts the skin and flesh layers, opening the incision. Since the support arms 18 and 20 are positioned below the knee-joint, the force against the retracted skin and flesh is disposed along a downward slope from the point of retraction to either the support 18 or 20.

The attaching device 44 is secured to the support arm 18 through aperture 52 through which the support arm 18 extends. To prevent the attaching device from rotating about the support arm 18, the support arm 18 includes a flat section 54 that cooperates with or acts against a flat or straight section 56 of the aperture 52. The support arm 20 also has a like flat section (not shown) for the same purpose. It should be understood by those skilled in the art that other methods of preventing rotation of the attaching device 44 about the arm 18 are included within the scope of the present invention. Such other methods of retaining the attaching device 44 may include clamps, set screws, pins and the like.

The attaching device 44 extends in a direction generally away from the incision 30 and has a distal end 58 that includes a V-shaped notch 60. When the flexible connector 42 is pulled back, a free end 43 of the connector is inserted between opposing sides of the V-shaped notch 60 for engagement. The V-shaped notch 60 pinches the flexible connector 42, thereby holding or retaining the flexible connector 42 in a pinched or frictional engagement.

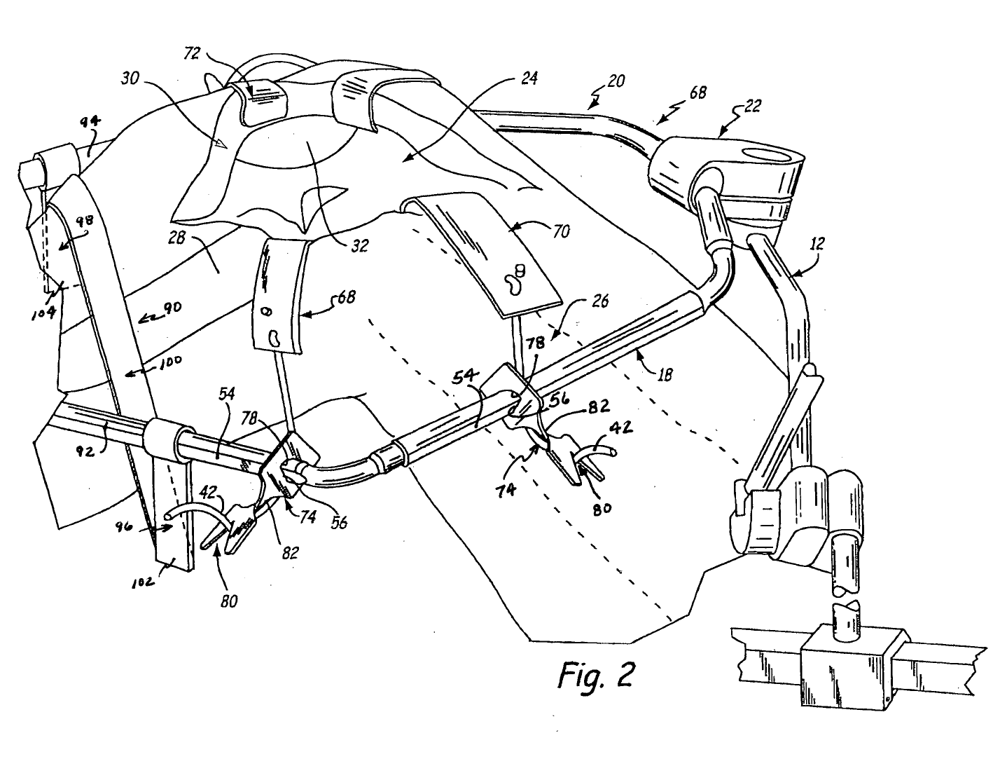

The surgical procedure of the present invention can be performed entirely using the retractor with flexible connectors of the present invention as illustrated in FIG. 2, where like reference characters will be used to indicate like elements of FIG. 1. The apparatus generally indicated at 68 includes retractors 68, 70, 72 and 73, all which have flexible connectors 42. All are mounted to either one of the support arms 18 and 20 which in turn is mounted to the rail 11 of the surgical table 13.

The flexible connector 42 of the surgical retractor 68, 70, 72 and 73 is attached to the support arms 18 and 20 by an attaching device 74 that has a different configuration than the attaching device 44 illustrated in FIG. 1. However, the attaching device 74 is secured to the support arms 18 and 20 in a similar fashion, and that is by an aperture 76 having a flat section 78 that cooperates or acts against the flat section 54 of the support arm 18. Similarly, the flexible connector 42 is engaged in a V-shaped notch 80 similar to the V-shaped notch 60 of the attaching device 44. The primary difference between the attaching device 74 and the attaching device 44 illustrated in FIG. 1 is that the attaching device 74 is made of flat sheet metal wherein the mid-section 82 of the device 74 is twisted approximately 90° to provide rigidity to the attaching device. Rigidity is provided to the attaching device 44 by virtue of its arcuate cross-section.

A lower leg hold down device 90 is attached to distal end portions 92 and 94 of the support arms 18 and 20, respectively. The hold down device 90 includes a pair of downwardly extending rigid legs 96 and 98 that project downwardly from the distal end portions 92 and 94. A flexible strap 100 is attached at both ends to lower portions 102 and 104 of the downwardly extending legs 96 and 98, respectively. The legs 96 and 98 are secured in a rigid fashion to the distal end portions 92 and 94 of the support arms 18 and 20. The flexible strap 100 is attached to the downwardly extending legs at a position below the point at which the strap engages the lower leg so that a force is applied to the lower leg to retain the lower leg in position. Holding the lower leg down in position eliminates the need for manual retention of the lower leg during surgery or the use of other additional devices that may be secured to the working surface of the surgical table.

After the retractors 68, 70, 72 and 73 are positioned to retract skin and flesh, the patella 32 is either removed or moved aside thereby exposing the ends of the femur and the tibia. Since the knee-joint 24 is in a bent position, the end of the femur is accessible to the surgeon through the incision. Due to the flexible connector of the retractors, the knee-joint can be repositioned without having to adjust the position of the support arm 18 or 20 or readjusting the retractor blade or reattaching the retractor blade to either support arm or both, 18 and 20. The end of the femur is then cut, and prepared to accept a prosthetic insert made of metal such as stainless steel or titanium as is standard in knee-joint replacement.

After the end of the femur has been prepared to accept the prosthetic insert (not shown), the tibia must then also be prepared to accept a prosthetic insert typically made of polyethylene which interacts with the first prosthetic insert of the femur. To prepare the tibia to accept the second prosthetic insert, the tibia must be pushed away from the femur and lifted to gain access to the end of the femur. Since the retractors of the present invention have flexible connectors, the tibia may be moved with respect to the femur without the need to reposition retractor blades or reattach the retractors to the support arms 18 or 20 or adjust the support arms 18 or 20. Once the surgeon cuts the end of the tibia and secures the second prosthetic insert to the tibia, the tibia with prosthetic insert is then maneuvered to engage the prosthetic insert on the femur.

Once the two prosthetic inserts are engaged, the surgical retractor of the present invention are disengaged from their retracted position. The patella is then moved back in position or a new prosthetic patella is substituted and the surgery is then completed.

Although the present invention has been described with reference to preferred embodiments, workers skilled in the art will recognize that changes may be made in form and detail without departing from the spirit and scope of the invention.

What is claimed is:

1. A method of performing a knee-joint replacement surgery involving a tibia and femur, the method comprising:
positioning the tibia and the femur such that the knee-joint is disposed in a bent position;
incising skin and flesh layers to expose the knee-joint;
positioning a retractor support extending along at least opposite sides of the knee joint;
retracting the skin and flesh layers proximate the knee-joint utilizing a plurality of retractors attached to the retractor support with at least one of the retractors being attached to the retractor support such that the retractor may be flexed sufficiently to be able to move the kneejoint during the surgery without repositioning the retractors.

2. The method of claim 1 wherein the retractor support is mounted to the surgical table.

3. The method of claim 2 wherein the retractor support is mounted to a rail of the surgical table.

4. The method of claim 1 wherein the at least one of the retractors is attached to the retractor support with a flexible cord.

5. The method of claim 4 wherein the flexible cord is attached to the retractor support by engaging a member that is rigidly attached to the retractor support, the member having a V-shaped groove wherein the cord engages the groove.

6. The method of claim 1 wherein the retractor support includes two support arms, each arm extending along an opposite side of the knee-joint.

7. The method of claim 6 and further including positioning the support arms below the knee-joint.

8. The method of claim 1 wherein each of the plurality of retractors are attached to the retractor support in a manner that each of the retractors may be flexed so that the knee joint may be moved during surgery without repositioning the retractors.

9. An apparatus for use in performing a knee-joint replacement surgery on a surgical table, the surgery involving a tibia and femur, the apparatus comprising:
a retractor support rigidly mounted to the surgical table; and
a plurality of retractors attached to the retractor support and each having a retractor blade for engaging skin and flesh layers proximate the knee-joint wherein at least one of the retractors has a flexible portion that flexes sufficiently so that the tibia may be moved during the surgery without having to re-engage the retractors with the flesh and skin layers or to reattach the retractors to the retractor support or adjust the retractor support.

10. The apparatus of claim 9 wherein the surgical table includes a rail and wherein the retractor support is rigidly mounted to the rail of the surgical table.

11. The apparatus of claim 9 wherein the at least the one of the retractors includes a flexible cord attached to the retractor blade, the cord having a free end that is securable to the retractor support.

12. The apparatus of claim 11 and further including an attaching device fixedly attached to the retractor support and having a V-shaped groove engaging the free end of the cord.

13. The apparatus of claim 9 wherein the retractor support includes at least two arms, each arm positioned along opposite sides of the knee joint.

14. The apparatus of claim 13 wherein the arm of the retractor support are positioned below the knee joint.

15. The apparatus of claim 9 wherein each of the plurality of retractors has a flexible portion which flexes sufficiently to move the tibia during surgery without having to re-engage the retractors with the flesh and skin layers or reattaching the retractors to the retractor support or adjust the retractor support.

16. The apparatus of claim 15 wherein each of the retractors includes a flexible cord having a free end that is attachable to the retractor support.

17. A method of holding down a lower leg portion during knee surgery, the method comprising:
positioning the knee such that the knee is in an elevated and bent position facing generally upwardly;
positioning a retractor support mounted to a surgical table, the retractor support having first and second arms that extend along opposite sides of the knee; and
positioning a flexible strap extending between distal ends of the first and second arm such that the flexible strap engages the lower leg with a downward force to retain the lower leg in a selected position during surgery.

18. The method of claim 17 wherein the retractor support is mounted to a rail of the surgical table.

19. An apparatus for use in securing a lower leg in knee surgery on a surgical table wherein the knee is disposed in an elevated position, the apparatus comprising:
a retractor support mounted to the surgical table wherein the retractor support includes first and second support arms that extend along opposite sides of the knee, each support arm having distal end portions; and
a flexible strap extending between the distal end portions of the support arm for engaging the lower leg such that a generally downward force is applied against the lower leg to retain the lower leg in position.

20. The apparatus of claim 19 and further including first and second downwardly extending rigid leg portions extending from the first and second distal end portions of the first and second support arms and wherein the strap is fixedly attached to the first and second leg portions.

21. The apparatus of claim 20 wherein the strap is attached to the first and second leg portions below where the strap engages the lower leg.
